(19) United States
(12) Patent Application Publication
(10) Pub. No.: US 2022/0167850 A1
(43) Pub. Date: Jun. 2, 2022

(54) 3D Body Scanner for Generating 3D Body Models

(71) Applicant: Naked Labs Austria GmbH, Wien (AT)

(72) Inventors: Gerhard Schultes, Wien (AT); Peter Kreuzgruber, Wien (AT)

(21) Appl. No.: 17/436,788

(22) PCT Filed: Mar. 6, 2020
(86) PCT No.: PCT/EP2020/056021

§ 371 Date: Sep. 7, 2021

(30) Foreign Application Priority Data
Mar. 8, 2019 (AT) A 60062/2019

(51) Int. Cl. A61B 5/00; A61B 5/0507; G01D 21/02; G01B 15/00

(52) U.S. Cl. CPC A61B 5/0064; A61B 5/0022; A61B 5/002; G01B 15/00; A61B 5/0077; G01D 21/02; A61B 5/0507

(57) ABSTRACT

A 3D body scanner for generating 3D body models includes a first device that includes a depth sensor for acquiring depth data of a field of view of an object to be scanned. The 3D body scanner includes a first communication interface and a control unit, which is alternatively configured for processing the depth data. The 3D body scanner includes a second device that includes a sensing component for detecting the object to be scanned. The second device is designed for sending to the control unit, an activation signal after detecting the object to be scanned. The control unit is configured to activate at least the first device upon the acquisition of the depth data.

### CROSS REFERENCE TO RELATED APPLICATIONS

The present application claims priority to application Serial No. PCT/EP2020/056021 filed Mar. 6, 2020, which is hereby incorporated herein in its entirety by this reference for all purposes.

### FIELD OF THE INVENTION

The invention concerns a 3D body scanner for generating 3D body models and includes a depth sensor for acquiring depth data of a field of view, a first communication interface, and a control unit for controlling the 3D body scanner and/or processing the depth data.

### BACKGROUND OF THE INVENTION

To generate 3D human body models 3D body scanners are used. These are complicated gadgets composed by several optical, mechanical and electronic sub-systems. If not in active scanning operation, all parts of 3D body scanners are either fully turned off or, more usually in a very low power standby mode.

Today accurate 3D human body models are demanded by various fields of applications as are, for example, fitness and body styling application, medical applications, cloth manufacturing industry, cloth internet—and retail shops and/or automotive industry.

Known 3D body scanners measure the body dimensions while the body rotates on a turntable or, at recent developments, by its own motion. Prior to scanning, all scanner components, which may include for example the scanner mast with depth sensor(s) and camera(s), the turntable with motor and/or possibly scale, a computation unit(s), the user interface and communication interfaces between these units, have to be activated.

The 3D home scanner is scanning while the user is standing, for example, on a turntable, which is in most cases mechanically separated from a scanner mast. The 3D home scanner is activated by an external device such as smartphone or tablet which should be placed somewhere remote from the user during scanning. Also, here some of the following consecutive user interactions for starting a scan are required: Unlocking smartphone. Starting body scanner app. Starting scan. Placing the smartphone somewhere besides. Stepping on turntable.

Also, here similar consecutive user interactions (not considering a scale) for starting a scan are required: Unlocking smartphone. Starting body scanner app. Starting scan. Placing the smartphone somewhere besides. Moving to correct scanning position.

In this case, the disadvantage is the time between manual scanner activation and the real start of scanning process, especially when the 3D body scanner is out of hand reaching distance. This frequently leads to the cases that first or last part of the defined scanning motion is lost.

### OBJECTS AND SUMMARY OF THE INVENTION

An object of the disclosed invention is to provided 3D body scanners that are configured to lend themselves to being activated in convenient ways with very simple user interaction or without any user interaction.

It is proposed that a 3D body scanner for generating 3D body models should include a first device comprising a depth sensor for acquiring depth data of a field of view and a first communication interface. The 3D body scanner comprises further a control unit for controlling the 3D body scanner and/or processing the depth data. With the help of the control unit the 3D body model can be generated out of the depth data.

Furthermore, the 3D body scanner comprises a second device for a user to be scanned, wherein the second device further comprises a sensing component for detecting the user to be scanned and wherein the second device is designed in such a way that it can send an activation signal after detecting the user to the control unit that is further designed in such a way that it can activate at least the first device, in particular the acquiring of the depth data. Therefore, the second device can activate the first device and in particular the acquiring of the depth data. Therefore, no manual activation of the acquiring of the depth data is necessary.

In an advantageous embodiment of the 3D body scanner, the first device comprises the control unit.

In an advantageous embodiment of the 3D body scanner, the second device comprises a second communication interface and wherein the activation signal can be sent via the second communication interface to the first communication interface.

In an advantageous embodiment of the 3D body scanner, the first communication interface and/or the second communication interface is a wireless communication interface, in particular WiFi, Bluetooth, WLAN or a light emitter and a light receiver. Alternatively, the first communication interface and/or the second communication interface is a wired communication interface, in particular USB, LAN or a power supply line.

In an advantageous embodiment of the 3D body scanner, the sensing component of the second device can detect a disconnecting of a power supply, in particular a power supply connection with the first device. Therefore, the sensing component of the second device can detect a disconnecting of the power supply line between the first and the second device.

In an advantageous embodiment of the 3D body scanner, the first device comprises a first power supply interface and the second device comprises a second power supply interface, wherein the second device can be supplied and/or charged via the power supply interfaces with electrical energy.

In an advantageous embodiment of the 3D body scanner, the activation signal can be sent via the power supply interfaces.

In an advantageous embodiment of the 3D body scanner, the second device comprises a turntable and/or a scale for rotating, weighing and/or presenting the user to be scanned. The user can step on the second device for scanning of the body of the user.

In an advantageous embodiment of the 3D body scanner, the sensing components of the second device comprise a force sensor, an accelerometer, a gyroscope, a torque sensor, a capacity sensor, a conductivity sensor, a temperature sensor, a light strength sensor, a button, a switch and/or a power supply interface.

In an advantageous embodiment of the 3D body scanner, the sensing components of the second device can measure an amplitude and/or a direction of the detected parameter. For example, for the amplitude or the size of the parameter a weight of the user can be measured. Furthermore, a threshold of the amplitude can be defined at which the activation signal will be sent. For example, if the measured weight is in the range of 5 kilogram, the activation signal will not be sent. Maybe a pet stepped on the second device and the activation signal should not be sent.

In an advantageous embodiment of the 3D body scanner, the depth sensor is by stereo vision, assisted stereo vision, structured light, time of flight, Lidar and/or, in particular microwave, radar and/or wherein the first device further comprising an optical camera, in particular a RGB-camera, black-white camera and/or infrared camera.

In an advantageous embodiment of the 3D body scanner, the 3D body scanner comprises an external device that can communicate with the first device and/or with the second device via the first and/or the second communication interface and can in particular send the activation signal to the control unit.

Furthermore, it is proposed a method for operating a 3D body scanner for generating 3D body models. The 3D body scanner can be designed according to one or more of the features of the previous and/or following description. Alternatively, or additionally the 3D body scanner of the previous and/or following description can be designed in such a way that the 3D body scanner can perform a method according to one or more steps of the previous and/or following description.

The method comprises the step of detecting a user to be scanned by sensing components of a second device. The method further comprises the sending of an activation signal, in particular via a second communication interface of the second device, to a control unit and activating a first device for scanning the user to be scanned for generating 3D body models.

In an advantageous embodiment of the method the first device will be activated before the second device will be activated. This means that only the sensing components are active. After the detecting of the user the second device will be fully activated. After this the activation signal will be sent to the first device. Then the first device will be activated. This has the advantage that the second device can decide whether the activation signal will be sent.

### BRIEF DESCRIPTION OF THE DRAWINGS OF EXEMPLARY EMBODIMENTS

Additional advantages of the invention are described in the following exemplary embodiments. The drawings show in:

FIG. 1 a 3D body scanner with a user and

FIG. 2 a second device with a user.

### DETAILED DESCRIPTION OF EXEMPLARY EMBODIMENTS OF THE INVENTION

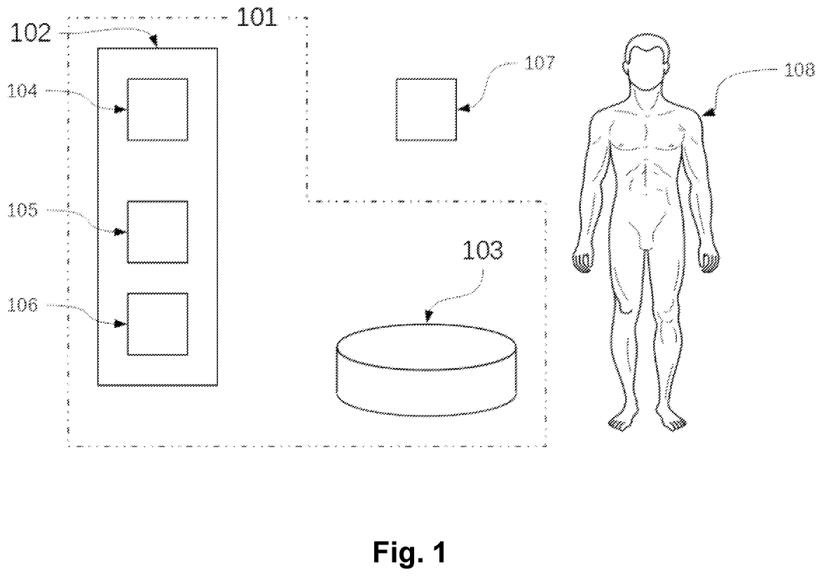

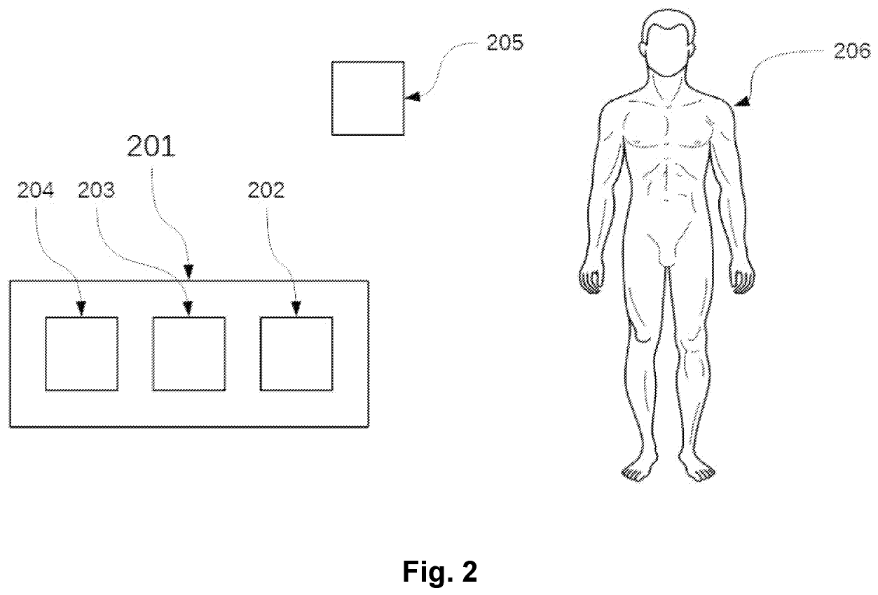

FIG. 1 U shows a 3D body scanner 101 within the chain-dashed outline and alongside a user 108. Furthermore, FIG. 2 shows a second device 103, 201 with the user 206. Note that for some features different references may be used. For example, the second device has the reference 103 in FIG. 1 and the reference 201 in FIG. 2. A 3D body scanner 101 consists of a first device 102 called scanning unit and contains a second device 103 which is a turntable and/or a scale. The second device 103 can be docked (connected) in some realizations to the first device 102 for energy supply and battery charging.

The first device 102 may be, for example, a large apparatus placed on the floor or a small unit installed hanging somewhere on the wall or standing on a furniture unit. The first device 102 may contain besides other functional devices, at least one of, but not limited to, in single or multiple number, the following sensing components 104: Depth sensor as the sensing components 104 realized for example, but not limited to: Stereo vision; ○Assisted stereo vision; ○Structured light; ○Time of flight; ○Lidar; ○Radar. Optical camera for example, but not limited to: ○RGB; ○BW; ○IR. Infra-red detector for example, but not limited to: ○Passive; and ○Active. Microwave radar for example, but not limited to: ○single beam; ○multi beam. Lidar for example, but not limited to: ○single beam; ○multi beam.

The first device 102 may contain besides other functional devices and sensing components 104, at least one of, but not limited to, in single or multiple number, the following first communication interfaces 105 for communication with the user 108, external devices 107 and/or the second device 103 if available: Mechanical communication interface: ○Button; ○Switch. Touch detector: ○Capacitive; ○Conductive; ○Temperature; ○Light. Acoustical communication interface, but not limited to: ○Microphone; ○Speaker. Wireless communication interface for example, but not limited to: ○WiFi; ○Bluetooth. Optical communication interface for example, but not limited to: ○Light emitter; ○Light receiver. Wired communication interface for example, but not limited to: ○USB.

The first device 102 may contain besides other functional devices, sensing components 104 and communication interfaces 105, at least one of, but not limited to, in single or multiple number, of the power supply interfaces 106 which may have at least limited communication capabilities too, for supplying or power charging of the second device 103 if available: Wired power supply unit for example, but not limited to: ○USB. Wired battery charging unit for example, but not limited to: ○USB. Wireless power supply unit for example, but not limited to: ○Standardized; ○Proprietary. Wireless battery charging unit for example, but not limited to: ○Standardized; ○Proprietary. Docking detector for supplying or charging for example, but not limited to: ○Mechanic; ○Capacitive; ○Magnetic. Solar battery charging unit.

The second device 103, 201 is an apparatus placed on the floor in visibility of the first device 102. Besides other data collection purposes, the second device 103, 201 can rotate the user 108 during the scan process. The second device 103, 201 may contain besides other functional devices, at least one of, but not limited to, in single or multiple number, the following second device sensing components 202: Scale (force sensor); Accelerometer: Gyroscope; Torque sensor of rotating surface (directly or indirect by the motor). Capacity sensor; Conductivity sensor; Temperature sensor; Light strength sensor.

The second device 103, 201 may contain besides other functional devices and second device sensing components 202, at least one of, but not limited to, in single or multiple number, the following: second communication interfaces 203 of the second device 103, 201 for communication with the user 108, the first device 102, the sensing unit and external devices 107: Mechanical communication interface: ○Button; ○Switch. Touch detector interface for example, but not limited to: ○Capacitive; ○Conductive; ○Temperature; ○Light. Acoustical communication interface, but not limited to: ○Microphone; ○Speaker. Wireless communication interface for example, but not limited to: ○WiFi; ○Bluetooth; ○Any other. Optical communication interface for example, but not limited to: ○Light emitter; ○Light receiver. Wired communication interface for example, but not limited to: ○USB.

The second device 103, 201 may contain besides other functional devices, sensing components 202 and communication interfaces 203, at least one of, but not limited to, in single or multiple number, of the second device power supply interfaces 204 which may have at least limited communication capabilities too, for being power supplied or power charged: Wired power supply unit for example, but not limited to: ○USB. Wired battery charging unit for example but not limited to: ○USB. Wireless power supply unit for example but not limited to: ○Standardized; ○Proprietary. Wireless battery charging unit for example but not limited to: ○Standardized; ○Proprietary. Docking detector for being supplied or charged: ○Mechanic; Capacitive; ○Magnetic. Solar battery charging unit.

If not in use, the first device 102 is typically in stand by mode. This means all components and functions which are not needed are turned off. Only the communication interfaces 105, sensing components 104 or, if available, power supply interfaces 106 used or intended to detect that a user wants to start a scan are, if possible, in advantageous way, in a low duty cycled mode active or whether an activation signal will be sent to the first device 102. Which communication interfaces 105, sensing components 104 and/or power supply interfaces 106 are intended for activation and starting the scan process depend on the implemented activation concept, and the second device 103.

It is advantageous, if the activation signal will be sent from the second device 103, 201 to the first device 102. The first device 102 can be activated by the activation signal sent from the second device 103, 201.

The first device 102 of a 3D body scanner 101 can be activated for scanning in an advantageous way by the communication interfaces 105 if a direct user interaction is detected, and/or a command and/or message from an external device 107 is received.

The activation by communication interfaces 105 can be configured to be dependent upon which interfaces are available and active, for example operating a mechanical button or switch, touching of the touch detector(s), voice command(s) recorded by microphone(s), message(s) received from external device(s) 107 as are but not limited to an optical control unit, a smartphone, a laptop, or any other wireless control or a wired control unit, or any combination of such events.

When having a 3D body scanner 101 consisting of a first device 102 and also a second device 103, in one embodiment the first device 102 is activated via communication interface 105 which may be, but not limited to, WiFi, Bluetooth or any other wireless ow wired solution, by the second device 103.

Furthermore the first device 102 can be activated for scanning in an advantageous way without direct user interaction by the active sensing components 104 if the approach or presence of a user in range of the first device 102 is detected.

The activation by sensing components 104 can be configured to be dependent upon which sensing components are available and active, identification of a user by analysis of depth map (depth image), and/or optical image, for shape of a user or simply a change over time, by detection of the approach of a user by its own infrared radiation or its reflection of microwave—, infrared—or any other radiation, or any combination of such events.

Furthermore if the optional second device 103 is available, the first device 102 can be activated for scanning in an advantageous way by indirect user interaction reported from the active power supply interfaces 106 if a preparation action for scanning as is for example, but not limited to moving the second device 103 from charging to the scanning position by the user is detected.

The activation by power supply interfaces 106 can take place if the second device 103, 201 is removed from its charging position close to or docked to the first device 102. For example, the sensing component 202 of the second device 103, 201 and/or the sensing component 104 of the first device 102 can detect the disconnecting of the power supply line. Such removing from its charging position can be in an advantageous way detected for example, but not limited to, by loss of galvanic connection, reduced wireless energy transfer or by a docking detector.

If not in use, the second device 103, 201 is typically in stand by mode. This means all components and functions which are not needed, are turned off. Only the second device communication interfaces 203, second device sensing components 202 and the second device power supply interfaces 204 needed to detect that a user wants to start a scan are, in advantageous way, active in a low duty cycled mode. Which second device communication interfaces 203, second device sensing components 202 and second device power supply interfaces 204 are used for activation and starting the scan process depends on the implemented wake-up concept and, if the second device 103, 201 is standing alone or put in a charging position in relation to the first device 102.

The second device 103, 201 of a 3D body scanner 101 can be activated in an advantageous way by the active second device communication interfaces 203 if a user interaction is detected, and/or a command and/or message from an external device 205 and/or by the second device power supply interfaces 204 if the charging supply is interrupted and/or an activation message is received over the second device power supply interfaces 204.

The activation by second device communication interfaces 203 can be configured to be dependent upon which interfaces are available and active for example, operating a mechanical button or switch, touching of the touch detector(s), voice command(s) recorded by microphone(s) or message(s) received from external device(s) 205 as are but not limited to an optical control unit, a smartphone, a laptop, any other wireless control or a wired control unit, or any combination of such events.

A case is that the second device 103, 201 is activated for scanning via second device communication interface 203 which may be, but not limited to, WiFi, Bluetooth or any other wireless or wired solution, by a command from the first device 102.

Furthermore the second device 103, 201 can be activated for scanning in an advantageous way without direct user interaction by the active second device sensing components 202 if the approach to—or presence of a user on the second device 103, 201 is detected.

The activation by second device sensing components 201 can be configured to be dependent upon which sensing components are available and active, for example value of force or change of force caused by mass or change of mass at step on the second device 103, 201, acceleration or rotation at step on the second device 103, 201, by moving or shifting the second device 103, 201 in correct scanning position, applying torque on the rotating part of the second device 103, 201 at step on the second device 103, 201, or changing the capacity, conductivity or temperature or light strength at the surface of the second device by placing a load, or any combination of such events.

Furthermore the second device 103, 201 can be activated for scanning in an advantageous way by indirect user interaction from the active second device power supply interfaces 204 if a preparation action for scanning as is for example, but not limited to moving the second device 103, 201 from charging to the scanning position by the user is detected.

The activation by second device power supply interfaces 204 can take place if the second device 103, 201 is removed from its charging position close to or docked to the first device 102. Such removing from its charging position can be detected by loss of galvanic connection, reduced wireless energy transfer or by the docking detector. Therefore, the power supply line is interrupted and the sensing component 202 of the second device 103, 201 can detect this. After this, the activation signal can be sent to the first device for activation.

Alternatively. it is comfortable for the user that the second device 103, 201 is activated at first then this activates the first device 102 via communication interfaces 105, 203 and possibly, when connected (docked) to the first device 102 or in communication range of the by power supply interfaces 106, 204, by the power supply interfaces 106, 204.

It is also advantageous for the user that the first device 102 and the second device 103, 201 are independently activated directly or indirectly by the user 108, 206 or by external devices 107, 205 and establish a mutual communication for, but not limited to, synchronization of scanning actions and/or data transmission via communication interfaces 105, 203 or possibly, when connected (docked) to the first device 102, or in communication range of the by power supply interfaces 106, 204 by the power supply interfaces 106, 204.

The invention is not limited to the embodiments shown or described. Rather, any and all combinations of the individual features described, as shown in the figures or described in the description, and to the extent that a corresponding combination appears possible and sensible, are subject matters of the invention.

An advantageous feature is that the first device 102 of a 3D body scanner 101 can be activated for scanning in an advantageous way by the communication interfaces 105 if a direct user interaction is detected, and/or a command and/or message from an external device 107 is received.

An advantageous feature is that the activation of the first device 102 by communication interfaces 105 can be, in an advantageous way, dependent which interfaces are available and active for example, but not limited to, operating a mechanical button or switch, touching of the touch detector(s), a voice command recorded by microphone(s), message(s) received from external device(s) 107 as are but not limited to, an optical control unit, a smartphone, a laptop, or any other wireless control or a wired control unit, or any combination of such events.

An advantageous feature is that the 3D body scanner 101 consists of a first device 102 and also a second device 103, that the first device 102 is activated via communication interface 105 which may be, but not limited to, WiFi, Bluetooth or any other wireless or wired solution, by the second device 103.

An advantageous feature is that the 3D body scanner can be activated for scanning in an advantageous way without direct user interaction by the sensing components 104, if the approach or presence of a user in range of the first device 102 is detected.

An advantageous feature is that the activation of the first device 102 by sensing components 104 can be in an advantageous way, dependent which sensing components are available and active, for example, but not limited to, identification of a user by analysis of depth map (depth image), and/or optical image, for shape of a user or simply a change over time, by detection of the approach of a user by its own infrared radiation or its reflection of microwave—, infrared—or any other radiation, or any combination of such events.

The 3rd feature is, if the optional second device 103 is available, that the first device 102 of a 3D body scanner can be activated for scanning in an advantageous way by indirect user interaction reported from the active power supply interfaces 106 if a preparation action for scanning as is for example, but not limited to, moving the second device 103 from charging to the scanning position by the user is detected.

An advantageous feature is that activation of the first device 102 by power supply interfaces 106 can take place in an advantageous way, if the second device 103, 201 is removed from its charging position close to or docked to the first device 101 and such removing from its charging position is detected for example, but not limited to, by loss of galvanic connection, reduced wireless energy transfer or by the docking detector.

An advantageous feature is that the second device 103, 201 of a 3D body scanner 101 is activated in an advantageous way by the active second device communication interfaces 203 if a user interaction is detected, and/or a command and/or message from an external device 205 and/or by the second device power supply interfaces 204 if charging supply is interrupted and/or an activation message is received over the power supply interfaces 204.

An advantageous feature is that the activation of the second device 103, 201 by second device communication interfaces 203 can be configured to be dependent upon which interfaces are available and active, in an advantageous way, for example, but not limited to, operating a mechanical button or switch, touching of the touch detector(s), voice command(s) recorded by microphone(s) or message(s) received from external device(s) 205 as are for example, but not limited to an optical control unit, a smartphone, a laptop, any other wireless control or a wired control unit, or any combination of such events.

An advantageous feature is that the second device 103, 201 is activated for scanning in an advantageous way, via communication interface 203 which may be for example, but not limited to, WiFi, Bluetooth or any other wireless or wired solution, by a command from the first device 102.

An advantageous feature is that the 3D body scanner 101 can be activated for scanning in an advantageous way without direct user interaction by the active second device sensing components 202 if the approach of a user to—or presence of a user on the second device 103, 201 is detected.

An advantageous feature is that the activation of the second device 103, 201 by second device sensing components 201 can be, in advantageous way configured to be dependent upon which sensing components are available and active for example, but not limited to, value—or change of force caused by mass—or change of mass at step on the second device 103, 201, acceleration or rotation at step on the second device 103, 201 by moving or shifting the second device 103, 201 in correct scanning position, applying torque on the rotating part of the second device 103, 201 at step on the second device 103, 201 or, changing the capacity, conductivity or temperature or light strength at the surface of the second device by placing a load, or any combination of such events.

An advantageous feature is that the second device 103, 201 of a 3D body scanner 101 can be activated for scanning in an advantageous way by indirect user interaction from the active second device power supply interfaces 204 if a preparation action for scanning as is for example, but not limited to, moving the second device 103 from charging to the scanning position by the user is detected.

An advantageous feature is that the activation of the second device 103, 201 by second device power supply interfaces 204 can take place in an advantageous way, if the second device 103, 201 is removed from its charging position close to or docked to the first device 102, and such removing from its charging position can be detected by for example, loss of galvanic connection, reduced wireless energy transfer or by the docking detector.

An advantageous feature is that the 3D body scanner 101 consists of a first device 102 and a second device 103, 201, it is comfortable for the user that if the first device 102 is activated at first, then this activation of the first device 102 will then activate the second device 103, 201 via communication interfaces 105, 203 and possibly, when connected (docked) to the first device 102 or in communication range of the by power supply interfaces 106, 204, by the power supply interfaces 106, 204.

An advantageous feature is that the 3D body scanner 101 consists of a first device 102 and a second device 103, 201, it is comfortable for the user that if the second device 103, 201 is activated at first, then this activation of the second device 103, 201 activates the first device 102 via communication interfaces 105, 203 and possibly, when connected docked to the first device 102 or in communication range of the by power supply interfaces 106, 204, by the power supply interfaces 106, 204.

An advantageous feature is that the 3D body scanner 101 consists of a first device 102 and a second device 103, 201, it is also advantageous for the user that the second device 103, 201 and first device 102 are independently activated directly or indirectly by the user 108, 206 or external devices 107, 205 and establish a mutual communication for, but not limited to, synchronization of scanning actions and/or data transmission via communication interfaces 105, 203 or possibly, when connected (docked) to the first device 102, in communication range of the by power supply interfaces 106, 204 by the power supply interfaces 106, 204.

The 3D body scanner consists of a first device, for example a scanning unit, and may optionally also comprise a second device, for example a personal scale and/or a turntable, which can be, in some realizations docked (connected) to the first device for energy supply and battery charging. Common disadvantage of the state-of-the-art solutions is that they need a lot of preparation until the scan is started. The present invention discloses methods for an advantageous automatic activation of such a 3D body scanner from low-power stand by mode to scanning operation by detecting the approach to—or presence of the user in—the scan area, the stepping on the optional second device or moving this second device in correct scanning position.

1. 3D body scanner for generating 3D body models comprising:
a first device comprising a depth sensor and a first communication interface, wherein the depth sensor is configured for acquiring depth data of a field of view;
a control unit for controlling the 3D body scanner and/or for processing the depth data; and
a second device for a user to be scanned, wherein the second device further comprising a sensing component for detecting the user to be scanned and wherein the second device is designed in such a way that it can send an activation signal after detecting the user to the control unit that is configured to activate at least the first device.

2. 3D body scanner according to claim 1, wherein the first device comprises the control unit.

3. 3D body scanner according to claim 1, wherein the second device comprises a second communication interface and wherein the activation signal can be sent via the second communication interface to the first communication interface.

4. 3D body scanner according to claim 1, wherein the first communication interface and/or the second communication interface is a wireless communication interface, in particular WiFi, Bluetooth, WLAN or a light emitter and a light receiver, or a wired communication interface, in particular USB, LAN or a power supply line.

5. 3D body scanner according to claim 1, wherein the sensing component of the second device can detect a disconnecting of a power supply, in particular a power supply connection with the first device.

6. 3D body scanner according to claim 1, wherein the first device comprises a first power supply interface and the second device comprises a second power supply interface, wherein the second device can be supplied and/or charged via the power supply interfaces with electrical energy.

7. 3D body scanner according to claim 1, wherein the activation signal can be sent via the power supply interfaces.

8. 3D body scanner according to claim 1, wherein the second device comprises a turntable and/or a scale for rotating, weighing and/or presenting the user to be scanned.

9. 3D body scanner according to claim 1, wherein the sensing components of the second device comprise a force sensor, an accelerometer, a gyroscope, a torque sensor, a capacity sensor, a conductivity sensor, a temperature sensor, a light strength sensor, a button, a switch and/or a power supply interface.

10. 3D body scanner according to claim 1, wherein the sensing components of the second device can measure an amplitude and/or a direction of the detected parameter.

11. 3D body scanner according to claim 1, wherein the depth sensor is by stereo vision, assisted stereo vision, structured light, time of flight, Lidar and/or, in particular microwave, radar and/or wherein the first device further comprises an optical camera, in particular a RGB-camera, black-white camera and/or infrared camera.

12. 3D body scanner according to claim 1, wherein the 3d body scanner comprises an external device that can communicate with the first device and/or the second device via the first and/or the second communication interface and can in particular send the activation signal to the control unit.

13. Method for operating a 3D body scanner that is configured for generating 3D body models, the method comprising the following steps:
detecting a user to be scanned by sensing components of a second device,
sending an activation signal via a second communication interface of the second device, to a control unit, and
activating a first device for scanning the user to be scanned for generating 3D body models.

14. Method according to claim 13 with the further step that the second device will be activated before the first device will be activated.

15. 3D body scanner according to claim 1, wherein the control unit is configured to activate the first device to acquire the depth data.
